(19) United States
(12) Patent Application Publication
(10) Pub. No.: US 2003/0072687 A1
(43) Pub. Date: Apr. 17, 2003

(54) System to transport goods at consistent temperatures

(76) Inventors: Dirk Nehring, Langgoens (DE); Kai Bienert, Wettenberg (DE)

Correspondence Address: DIRK NEHRING, VERSAILLER STR. 1, GIESSEN, HESSEN, 35394, DE

(21) Appl. No.: 10/022,406

(22) Filed: Dec. 20, 2001

(30) Foreign Application Priority Data
Oct. 10, 2001 (DE) 01 124 070.2

(51) Int. Cl. C12M 3/00

(52) U.S. Cl. 422/102; 422/99; 422/82.12; 435/283.1; 435/288.1

(57) ABSTRACT

The invention is a system for the transport of products at a consistent temperature by use of a protective container for fragile products, specifically a container that protects and transports blood and medical products, with properties described in the detailed description section of this patent application.

### BACKGROUND OF THE INVENTION

The transport and storage of blood products requires a consistent cool chain in the temperature ranges of 2 to 6° C., <−20° C. and between 20° to 24° C. in order to maintain its life giving properties. To satisfy these requirements, blood products are transported in temperature regulated containers which are either passively cooled by means of accumulators or by active electrical cooling methods.

Such transportation containers are extensively used in the professional transportation of blood. A weakness in existing transport systems is however, in the un- and reloading of blood products, as they are passed over to the reciever in containers with only short term isolation properties. The adherence to the cool chain is, in such cases, mostly not quantifyable and the period in which the required temperatures are maintained is very short.

A further problem is with the internal transport in clinics. Blood products that, for example, leave storage in preparation for the operation theatre, are not kept under temperature control as they are presently transported in simple insulated containers. If this blood product is then not used, it is not quantifyable whether the permitted temperature levels have been exeeded. In order to save and re-store this blood a complicated and extensive analytical test must be undertaken. Furthermore, most containers used to transport blood do not have any protective mechanism against tampering.

In order to create a cool chain, previous methods used to transport temperature sensitive products at the required temperature levels have been to use insulated chambers (i.e. boxes) that has an insulated inner layer together with a simple cooling device that is filled with a liquid of high specific enthalpy. A disadvantage in using this method is however, that the liquid with high specific enthalpy comes into direct contact with the packaging in which the blood is kept and adherence to the required temperature levels is not visible from the outside. Furthermore, the thermal enthalpy of the fluid is not effective enough.

The purpose of this invention is to provide a transport system for products at consistent temperatures, specifically blood and medical products, which hinders tampering of single or multiple units and maintains the permitted temperatures. A further purpose of this invention is to show on the container whether the permitted temperatures have been maintained.

The solution is found with a system to transport products at consistent temperatures, specifically the temperature regulated transport of blood and medical products with the properties according to claim 1. Andvantageous properties of the invention are described in full in the claim.

### SUMMARY OF THE INVENTION

In accordance with the invention the system, a storage, protection and transport container for easily perishable products, made from double walls that are filled with a fluid or solid latent temperature regulating fluid to give it insulating properties, enables the transport of products at consistent temperatures, especially to transport blood and medical products.

A major property of the invention is that the container, consisting of two shells that close together, snap tightly shut due to a series of notches and indentations along the perimeter of each half. When the two shells are closed together a protective space is created within, which enables the storage of blood and/or pharmaceuticals.

The storing, protection and transporting system of the present invention comprises:

(1) two double walled shells with indentations and notches along the perimeter edges that allow them to fit tightly together and create an inner space for the storage and transport of blood, blood products or pharmaceuticals.

(2) A double walled ring with indentations and notches along the perimeter edges that allow it to fit tightly together with the two other shells to create a larger inner space for the storage of blood, blood products or pharmaceuticals.

(3) A contained (in the shells and ring) latent temperature regulating fluid, specifically paraffin, or a carrier immobilised paraffin, or a saline solution, or an ethanol water mix or a buthandiol water mix which provide a plateau of constant temperatures of −20° C. to −40° C., 2° C. to 6° C. and 20° C. to 24° C.

(4) Eyelets on each shell to allow the container to be sealed with a lead seal to stop undesired or at least recognisable opening of the container

(5) Grooves on each shell to allow for a carrying strap to be fitted.

(6) A sealed opening on each shell to allow them to be filled with the latent temperature regulating fluid.

(7) Each shell is fitted inside with a thermometer, specifically a self adhesive strip thermometer or a liquid crystal thermometer and/or an electronic readable temperature gauge.

### DESCRIPTION OF THE DRAWINGS

Other properties, peculiarities and advantages of the invention are shown in diagrammatical form and described as follows:

FIG. 1: an innerview perspective of the storage, protection and transport container according to the invention.

FIG. 2: an outer view perspective of the part shown in FIG. 1 of the storage, protection and transport container according to the invention.

FIG. 3: a perspective view of the ring that fits together with the parts in FIG. 1 and FIG. 2 of the storage, protection and transport container according to the invention, and

FIG. 4: a perspective outer view of the storage, protection and transport container according to the invention.

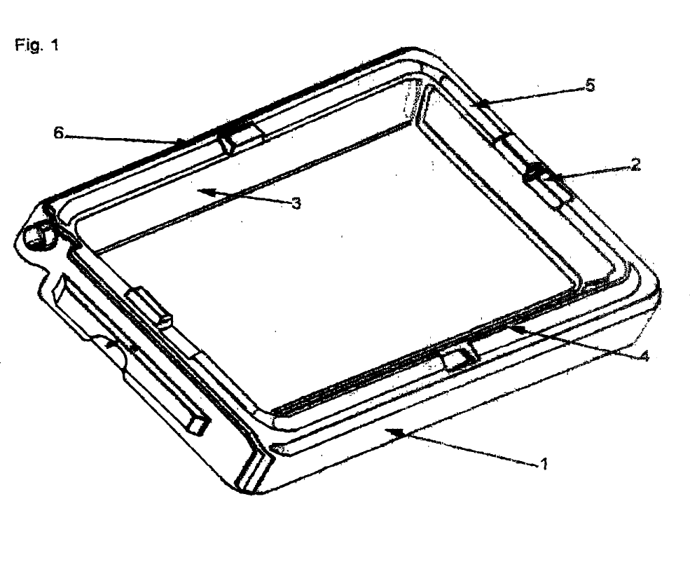

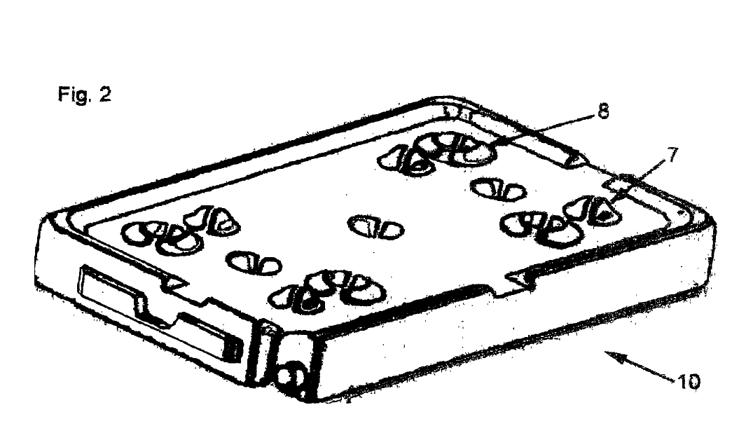

FIG. 1: The system to transport products at consistent temperatures consists of one shell, shown in diagram 1, which is locked together with a second same shell (FIG. 2) by means of indentations (shown as 2). The first shell locked together with a second shell creates an inner space (shown as 3), which is completely shielded from the outside elements due to the double walls of parts 1 and 10 and the tongue and grooves (shown as 4 and 5) along the connecting edges (shown as 6) of the container. This space (shown as 3) allows for the storage of blood products (not shown) or other products such as antibodies, cells or pharmaceuticals in their original packaging.

FIG. 2: Both parts 1 and 10 contain a liquid or solid temperature regulating medium within the double walls. The parts have indentations (shown as 8) on the outside and protrusions (shown as 7) to enable them to lock snugly together and allow for stacking. With the aid of a sealing tie(not shown) both parts can be sealed together.

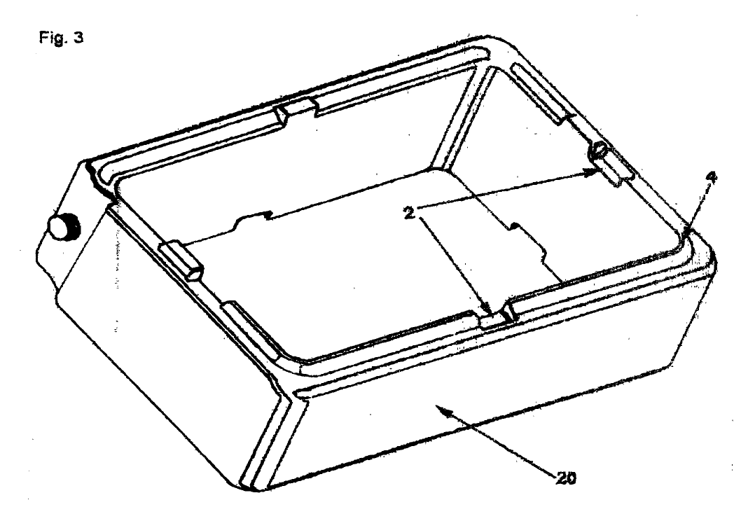

FIG. 3: Respective characteristics are marked in FIGS. 1 and 2. A double walled ring (part 20) fits together with parts 1 and 10 by means of tongues and grooves (4 and 5) along the connecting edges (shown as 6) thus increasing the interior volume.

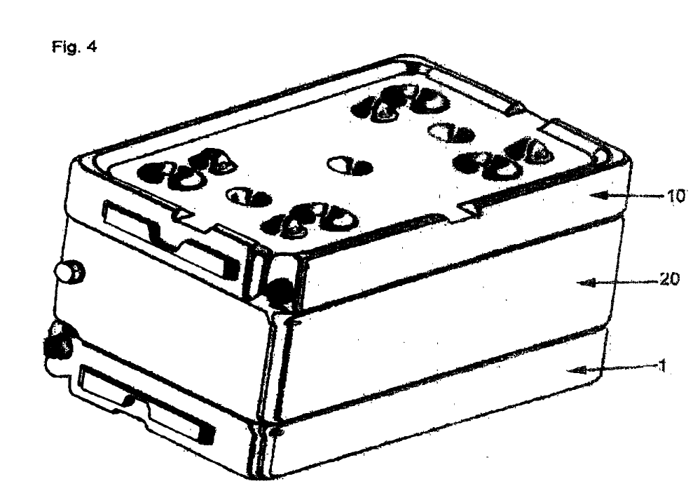

FIG. 4: Respective parts are marked in FIGS. 1, 2 and 3. The system used to transport products at consistent temperatures with improved protection and storage is achieved by fitting a shell (part 1) together with a second same shell (part 10) and a ring (part 20) which fits between both shells by means of grooves(shown as 2).

The double wall of parts 1, 10 and 20 all have sealable openings (not shown) which enable filling or emptying with a latent temperature regulating fluid, specifically paraffin or a carrier immobilised paraffin or a buthandiol water mix, a watery salt mix or an ethanol water mix, which has a melting point of either 20 to 24° C., 2 to 6° C., −20 to −30° C. and −20 to −40° C. Alternatively, n-paraffins with the formula C_{\mathring{5}}H _{\mathring{5}+} [symbol] [symbol] can be used to stabilise temperatures above 0° C.

The transparent walls of the storage, protection and transport container enables one to see the condition of the temperature regulating fluid and that the required temperatures have been adhered to. In this way blood products such as erythrocite concentrates and thrombocytes can easily be transported and handled at temperatures of 2 to 6° C. and 20 to 24° C.

The container, made up of 3 double walled shells which fit together to create an inner space for the products (shown as 3) in which the temperature should remain constant, is fitted with a thermometer (not shown), specifically a self adhesive strip thermometer, a liquid crystal thermometer and/or an electronic readable temperature gauge which can be set to read-temperatures of 20 to 24° C., 2 to 6° C., −20 to −30° and −20° C. to −40° C.

The container, made up of 2 or 3 complimentary double walled shells each have at least one eyelet (not shown) which allows the shells to be sealed together with a led seal, to prevent or show any undesired opening of it.

### DETAILED DESCRIPTION OF THE INVENTION

In accordance with the invention the system, a storage, protection and transport container for easily perishable products, made from double walls that are filled with a fluid or solid latent temperature regulating fluid to give it insulating properties, enables the transport of products at consistent temperatures, especially to transport blood and medical products.

A major property of the invention is that the container, consisting of two shells that close together, snap tightly shut due to a series of notches and indentations along the perimeter of each half. When the two shells are closed together a protective space is created within, which enables the storage of blood.

Another property of the invention is that both shells that fit together are double walled, which together with a similarly produced ring that also snaps onto the indentations, increases the protective storage space between them. In this way, the one shell serves as the top and the other as the bottom of the container. The double walled ring is also filled with a liquid, which according to the energy applied to it, may be charged with or emmit a required temperature.

Another property of the invention is that the double wall of each part is filled with a latent temperature regulating fluid which has a melting point of 2 to 6° C., specifically paraffin or a carrier immobilised parafin, which maintains the temperature of the storage space within at a consistent temperature of 2 to 6° C. The container is therefore capable of storing and transporting erythrocite concentrates and other blood products, antibodies, cells or biotechnically produced pharmaceuticalls which have to be transported at 2 to 6° C.

Another property of the invention is that n-hydrocarbons, namely n-paraffins with the formula CₙH₂ₙ₊₂ can be used to stabalise temperatures above 0° C.

Another property of the invention is that the container is filled with a saline solution or an ethanol water mix that has a consistency between fluid and solid between the temperatures of −20° C. and −40° C.

Another property of the invention is that the double wall of the container is filled with a latent temperature regulation fluid which has a melting point of 20 to 24° C., specifically paraffin Or a carrier immobilised paraffin or a buthanol water mix in order to keep the inner storage space at a constant temperature of 20 to 24° C. The container is therefore capable of storing and transporting thrombocytes and other blood products that have to be transported at these temperatures.

Another property of the invention is that the material from which the double walled container is produced is transparent, specifically a transparent plastic, so that the condition of the temperature regulating fluid within is visible. It is therefore easy to check the temperature levels making the container useful for cool chain transportation.

Another property of the invention is that the double walled shell is produced from impervious plastics such as polyamid e.g. kevlar, or from metals such as aluminium, steel or iron.

Another property of the invention is that the containers may be stacked as there are indentations on the back of the container allowing for this.

Another property of the invention is that there are two closures attached to the transport container that are sealable.

Another property of the invention is that the container is fitted with at least one eyelet which can be sealed so that undesired opening can be avoided or is at least recognisable.

Another property of the invention is that the parts of the storage, protection and transport container are fitted with tongue and groove to avoid temperature loss.

Another property of the invention is that the parts of the storage, protection and transport container are connested by a hinge.

Another property of the invention is that the container may be fitted with a carrying strap.

Another property of the invention is that the container is fitted inside with a thermometer, specifically a self sealing strip thermometer or a liquid crystal thermometer and/or an electronic readable temperature gauge.

What is claimed is:

1. A system to transport products at consistent temperatures, specifically blood products, antibodies, cells or biologically produced pharmaceuticals, by means of a container which stores and thus labels the product which is made from double walls filled with a temperature regulating fluid and maintains consistent temperatures.

2. The system according to claim 1, is thus characterised as the container is made up of two double walled shells which can be attached together by a clip along the longest sides of it.

3. The system according to claim 1, is thus characterised as the container is made up of two double walled shells and also a double walled complimentary ring, which can be attached together by a clip along the longest sides of it.

4. The system according to claim 1, is thus characterised as the container is filled with paraffin or a carrier immobilised paraffin as a temperature regulator.

5. The system according to claim 4, is thus characterised as the paraffin shows a condition between fluid and solid at temperatures between 2° C. and 8° C. or 20° C. and 24° C.

6. The system according to claim 4, is thus characterised as above 0° C. n-hydrocarbons can be used, namely n-paraffins with the formula CₙH₂ₙ₊₂.

7. The system according to claim 1, is thus characterised as the container is filled with a watery saline solution or an ethanol-water mix as a temperature medium which has a consistency between fluid and solid at temperatures between −20° C. and −40° C.

8. The system according to claim 1, is thus characterised as the container is filled with a buthandiol water mix as a temperature medium which has a consistency between fluid and solid at temperatures between 20° C. and 24° C.

9. The system according to claim 1, is thus characterised as the double wall is transparent, specifically made from a transparent plastic.

10. The system according to claim 1, is thus characterised as the double wall is made from an impervious plastic such as Kevlar, or from metals such as aluminium or iron.

11. The system according to claim 1, is thus characterised as the double wall has an outside locking mechanism.

12. The system according to claim 2, is thus characterised as the container consists of two double walled shells which can be sealed along the longer sides with a clip.

13. The system according to claim 3, is thus characterised as the container consists of two double walled shells and a complemetary double walled ring which can be sealed along the longer sides with a clip.

14. The system according to claim 11 or 12, is thus characterised as the container is fitted with an eyelet which allows for sealing.

15. The system according to claim 2, is thus characterised as the container made from two double walled shells that are fitted with tongues and grooves along the longer sides.

16. The system according to claim 2, is thus characterised as the container made from two double walled shells and a complementary double walled ring that are fitted with tongues and grooves along the longer sides.

17. The system according to claim 2 or 3, is thus characterised as the container made from two double walled shells and/or a complementary double walled ring are attached with a moveable hinge.

18. The system according to claim 2 or 3, is thus characterised as the container has a carrying strap.

19. The system according to claim 2 or 3, is thus characterised as the container made from two or three complementary double walled shells which enclose a space intended to store products is fitted with a thermometer, specifically a self adhesive strip thermometer or a liquid crystal thermometer and/or an electronicly readable temperature guage.
